(19) United States
(12) Patent Application Publication
(10) Pub. No.: US 2013/0203655 A1
(43) Pub. Date: Aug. 8, 2013

(54) METHODS FOR PREVENTING OR TREATING VIRAL INFECTION

(71) Applicants: Margaret Kielian, Pelham, NY (US); Aihua Zheng, Bronx, NY (US)

(72) Inventors: Margaret Kielian, Pelham, NY (US); Aihua Zheng, Bronx, NY (US)

(21) Appl. No.: 13/578,173

(22) PCT Filed: Feb. 9, 2011
(86) PCT No.: PCT/US11/00241

§ 371 Date: Sep. 5, 2012

Related U.S. Application Data
(60) Provisional application No. 61/337,957, filed on Feb. 12, 2010.

(51) Int. Cl. A61K 38/10

(52) U.S. Cl. CPC A61K 38/10
USPC 514/3.7; 435/5; 530/327

(57) ABSTRACT

The present invention provides methods to prevent or treat flavivirus infection, and assays for identifying agents which treat flavivirus infection. The present invention also provides compositions for preventing flavivirus infection and a kit for screening an agent that prevents or treats viral infection.

### CROSS-REFERENCE TO RELATED APPLICATIONS

This application claims benefit of U.S. Provisional Application No. 61/337,957, filed Feb. 12, 2010, the contents of which are hereby incorporated by reference.

### STATEMENT OF GOVERNMENT SUPPORT

This invention was made with government support under grant numbers R21-AI067931, RO1-GM057454, and 5U54AI057158 awarded by the National Institutes of Health, U.S. Department of Health and Human Services. The government has certain rights in the invention.

### FIELD OF THE INVENTION

The present invention relates generally to chemical strategies to screen for inhibitors of viral membrane proteins prM and E.

### BACKGROUND OF THE INVENTION

Throughout this application various publications are referred to in parenthesis. Full citations for these references may be found at the end of the specification. The disclosures of these publications are hereby incorporated by reference in their entirety into the subject application to more fully describe the art to which the subject invention pertains.

The emergence and resurgence of human viral pathogens can be traced to a complex variety of causes including increased urbanization, human contact with animal reservoirs, a decrease in effective public health systems, and the spread of insect vectors that disseminate some viral infections [1,2,3]. Flaviviruses are a genus in the Flaviviridae family and include important emerging and resurgent human pathogens such as dengue virus (DENV), West Nile virus (WNV), tick-borne encephalitis virus (TBEV) and yellow fever virus [2,4]. Flaviviruses are transmitted by insects such as mosquitoes and ticks, and can cause severe human diseases characterized by encephalitis, meningitis, and hemorrhages [2,3]. More than one third of the world's population lives in dengue fever endemic areas, and there are an estimated 50-100 million cases of dengue infection and 500,000 cases of the more lethal complication, dengue hemorrhagic fever, per year [5,6,7,8]. There are currently no antiviral therapies for flaviviruses. DENV vaccine development is underway but is problematic due to the presence of four DENV serotypes and the potential for antibody-dependent enhancement of infection [2,6,9,10]. Antiviral therapies could thus be an important alternative for DENV and for viruses such as WNV in which the cost and potential side effects of vaccination must be weighed against the relatively low number of human cases [2].

Flaviviruses are small, highly organized enveloped viruses with a spherical shape [4,11]. They contain a positive-sense RNA genome packaged by the viral capsid protein. The nucleocapsid is surrounded by a lipid bilayer containing the viral membrane protein E. Flaviviruses infect cells by receptor engagement at the plasma membrane, endocytic uptake, and a membrane fusion reaction triggered by the low pH of the endosome compartment [12,13]. The viral E protein binds the receptor and drives the fusion of the viral and endosome membranes to initiate virus infection. The pre-fusion structure of the E protein ectodomain (here referred to as E′) shows that E contains three domains composed primarily of β-sheets: a central domain I (DI) connecting on one side to the elongated domain II (DII) with the hydrophobic fusion loop at its tip, and connecting via a flexible linker on the other side to the immunoglobulin-like domain III (DIII) [14,15,16,17,18,19] (FIG. 1A). Although these regions are not present in the truncated E′ ectodomain, DIII connects to a stem domain and C-terminal membrane anchor (TM). The E protein in mature infectious flavivirus is organized in homodimers that lie tangential to the virus membrane [20]. Within each dimer the E proteins interact in a head to tail fashion, with the fusion loop of each E protein hidden in a hydrophobic pocket formed by DI and DIII of the dimeric E partner.

The E protein mediates virus-membrane fusion by refolding to a hairpin-like E homotrimer with the fusion loops and TM domains at the same end [21,22]. This reaction involves low pH-triggered dissociation of the homodimer, fusion loop insertion into the endosome membrane, formation of a core trimer composed of DI and DII, and the foldback of the DIII and stem regions towards the target membrane and their packing against the core trimer. The prefusion and postfusion conformations of the flavivirus E fusion protein are structurally and functionally similar to those of the El fusion protein from the alphavirus Semliki Forest virus (SFV) [23,24,25], and these fusion proteins are often referred to as “class II” [26,27,28]. In addition to the ectodomains whose trimer structures are described above, truncated fusion proteins composed of domains I and II (DI/II) can reconstitute SFV and DENV core trimer formation on target membranes [29,30]. Such core trimers act as specific targets for DIII binding, thus recapitulating the protein-protein interactions during class II trimerization and hairpin formation.

Flaviviruses bud into the endoplasmic reticulum (ER) and are transported as virus particles through the secretory pathway and released by exocytosis [4]. Given the low pH that is present in the Golgi complex and trans-Golgi network (TGN) [31], how do flaviviruses avoid inactivation during their transport? The particles are assembled in the ER as immature non-infectious viruses containing heterodimers of the precursor membrane protein (prM) and E protein [4,26,32]. Subsequent exposure to low pH in the secretory pathway triggers a dramatic rearrangement to E homodimers and makes the prM protein accessible to furin cleavage [33,34]. Processing of prM by cellular furin results in mature infectious virus in which E homodimers are poised to mediate fusion [33]. Important recent studies describe the structure of pr peptide in complex with E, and indicate that processed pr remains associated with the virus at low pH and can inhibit virus-membrane interaction [34,35,36]. Thus, pr on the virus could protect E protein from low pH in the secretory pathway.

The flavivirus prM/pr protein plays multiple roles in the virus life cycle [reviewed in 26]. prM acts as a chaperone for E protein folding [37] and associates with the tip of E [34]. prM also appears to respond to low pH to permit E rearrangement on the virus surface and allow furin access for prM processing [34,38]. Following cleavage, the pr peptide may prevent premature virus fusion through bridging interactions that stabilize the E homodimer and thereby prevent dissociation to E monomers, a key fusion intermediate [35,36]. The present invention provides methods to identify inhibitors against prM and protein E, which will inhibit virus infection by blocking viral lifecycle at these specific steps.

### SUMMARY OF THE INVENTION

A method is provided for treating or preventing infection by a flavivirus of a cell in a subject comprising administering to the subject an amount of an agent effective to (i) inhibit interaction of a pr peptide of the flavivirus and a membrane fusion protein E (E protein) of the flavivirus, or (ii) inhibit interaction between the E protein of the flavivirus and the membrane of the cell.

A method is provided for determining if an agent can prevent or reduce exocytosis from a flavivirus-infected cell of a flavivirus virus synthesized by the cell comprising:
a) contacting a membrane fusion protein E (“E protein”), or portion thereof, of the flavivirus with (i) a flavivirus pr peptide, and (ii) the agent under conditions permitting the pr peptide to bind to the E protein or portion thereof; and
b) quantifying the binding between the pr peptide and the E protein or portion thereof, wherein a decrease in the binding between the pr peptide and the E protein in the presence of the agent relative to binding between the pr peptide and the E protein in the absence of the agent indicates that the agent can prevent or reduce exocytosis of the flavivirus virus synthesized by the cell from the flavivirus-infected cell, while no change in or an increase in the binding between the pr peptide and the E protein in the presence of the agent relative to binding between the pr peptide and the E protein in the absence of the agent indicates that the agent is not useful to prevent or reduce exocytosis of the flavivirus virus from the flavivirus-infected cell.

A method is provided for determining if an agent can reduce or prevent fusion of a flavivirus with a cell membrane comprising:
a) contacting a membrane fusion protein E (“E protein”), or portion thereof, of the flavivirus with a flavivirus pr peptide and the agent under conditions permitting the pr peptide to bind to the E protein or portion thereof; and
b) quantifying the binding between the pr peptide and the E protein or portion thereof, wherein an increase in the binding between the pr peptide and the E protein in the presence of the agent relative to binding of the pr peptide and the E protein in the absence of the agent indicates that the agent can reduce or prevent fusion of the flavivirus with the cell membrane, while no change in or a decrease in the binding between the pr peptide and the E protein in the presence of the agent relative to binding between the pr peptide and the E protein in the absence of the agent indicates that the agent is not useful to reduce or prevent fusion of flavivirus with a cell membrane.

A peptide is provided comprising consecutive amino acid residues having the sequence TFKNPHAKKQDVVV (SEQ ID NO:4). In an embodiment the peptide is GCTFKNPHAKKQDVVVC (SEQ ID NO:5).

A pharmaceutical composition is provided comprising the peptide of claim 20 or 21 and a pharmaceutically acceptable carrier.

A peptide is provided comprising consecutive amino acid residues having the sequence TFKNPHAKKQDVVV (SEQ ID NO:4) for treating or preventing infection of a cell in a subject by a flavivirus. In an embodiment peptide is for treating or preventing infection of a cell in a subject by Dengue virus.

### BRIEF DESCRIPTION OF THE DRAWINGS

FIG. 1A-1C. Expression and purification of DENV2 pr and truncated E proteins. 1A) Linear diagrams of the DENV2 prM-E proteins and the truncated DENV2 pr and E proteins used in this work (not to scale). Domain and construct boundaries are marked, with numbering based on the individual proteins in the DENV2 New Guinea C (NGC) strain. The sequences appended to the diagrams contain the Strep (ST) affinity tag(s) used for protein purification (underlined), joined in the case of two Strep tags by a flexible linker region (STST). Pr was expressed in 293T cells and contains prM residues 1-86 plus N-terminal GS residues from the vector and the STST tag. The DI/II and E′ proteins were expressed in S2 cells and contain E residues 1-291 and 1-395, respectively, plus ST or STST tags. DIII was expressed in E. coli and contains E residues 289-430, comprising the linker, DIII, helix 1 and conserved sequence (LDIIIH1CS). The names in parentheses are the detailed nomenclature from [30]. 1B) 4 μg samples of purified pr peptide were incubated with DTT, Endo H, or PNGase F as indicated, analyzed by SDS-PAGE and stained with Coomassie blue. The positions of marker proteins are shown on the right with their molecular masses listed in kilodaltons. Asterisks indicate the positions of the added glycosidases. 1C) 4 μg samples of purified truncated E proteins were reduced with DTT as indicated, analyzed by SDS-PAGE and stained with Coomassie blue. Marker proteins are shown on the right with their molecular masses listed in kd. SEQ ID NOS.6-9, respectively for amino acid sequences attached to pr, DI/II (DI/II-STST) (top then bottom sequence), E′(E′-ST), and DIII (LDIIIH1CS).

FIG. 2A-2D. Pr peptide binds DENV E proteins in a pH-dependent manner. 2A) Pull-down of DI/II protein by pr. DI/II was incubated with sepharose beads conjugated with pr peptide or BSA at the indicated pH for 1 h at room temperature. As indicated, reactions contained a 2:1 molar excess of mAb 4G2 to the E fusion loop or mAb to the ST tag (con.). Input lanes show an aliquot representing 20% of the reaction prior to pull-down. (Panels 2B-2D) SPR analysis of pr-E binding. Pr peptide was immobilized on a CM5 sensor chip, and DENV2 E′ (2B), DI/II (2C) or SFV DI/II proteins (2D) were flowed over the chip at concentrations of 1.2 μM in buffers of the indicated pH for 300 s, followed by injection of protein-free buffer at the same pH. Data are a representative example of two independent experiments.

FIG. 3A-3E. Pr peptide inhibits E protein-membrane interaction. 3A) E′-liposome co-floatation assay. E′ protein was mixed with pr peptide or an ST-tagged control protein (Seap) at a final concentration of 50 μg E′ protein and 200 μg pr/Seap protein/ml (molar ratio 12 pr/1E). Liposomes were added at a final concentration of 1 mM, and the samples were incubated at the indicated pH for a total of 60 min at 28° C. Where indicated, E′ protein plus liposomes were incubated for 30 min, pr peptide added to a final concentration of 200 μg/ml, and the incubation continued for an additional 30 min. The liposome-bound proteins were then separated by floatation on sucrose gradients at the indicated pH. Aliquots of the top, middle and bottom of the gradients were analyzed by SDS-PAGE and western blotting for E protein. 3B) DI/II-liposome co-floatation assay. 40 μg/ml DI/II plus DIII or BSA (200 μg protein/ml) were incubated with liposomes plus 200 μg pr peptide/ml as indicated and assayed for liposome co-floatation as in panel 3A. 3C) SFV DI/II-liposome co-floatation assay. SFV DI/II protein (40 μg/ml) was mixed with BSA or pr peptide (160 μg/ml). Liposomes were added at a final concentration of 1 mM, and the samples were incubated at the indicated pH for a total of 30 min at 28° C. Liposome co-flotation was assayed as in panel 3A. 3D-3E) Loss of pr inhibition of E protein in the pH range of the late endocytic pathway. 3D) pH dependence of pr inhibition. E′ protein was mixed with liposomes in the presence or absence of pr peptide (molar ratio ˜12 pr/1E), treated at the indicated pH as in FIG. 3A, and E′-membrane association determined by floatation assay as in FIG. 3A. 3E) Concentration-dependence of pr inhibition. E′ protein was mixed with liposomes and treated at pH 5.75 or pH 5.0 in the presence of the indicated molar ratios of pr peptide to E protein. E′-membrane association was determined by floatation assay as in FIG. 3A. For each pH, the E′ floatation efficiency was normalized to the amount of floatation in the top fraction in the absence of added pr protein. Data in panels 3A-3E are each a representative example of two independent experiments.

FIG. 4. Pr peptide inhibits DENV fusion and infection. Serial dilutions of the indicated viruses were pre-bound to BHK cells by incubation for 90 min on ice at pH 7.9. The cells were then treated for 1 min at 37° C. in the presence of the indicated concentration of pr peptide using buffer at pH 6.0 to trigger virus fusion with the plasma membrane, or control buffer at pH 7.9. Cells were then incubated for 48 h in the presence of NH₄Cl to prevent secondary infection. Infected cells were quantitated by immunofluorescence, and the titers normalized to the pH 6.0 sample in the absence of pr. Each bar shows the average and range of duplicate wells. Representative example of two independent experiments.

FIG. 5A-5C. DENV E H244 is a key residue in pr-E binding. 5A) Sequence comparison of selected regions of the pr and E proteins from the 4 serotypes of DENV. The specific strains are DENV1 WP, DENV2 NGC, DENV3 H87 and DENV4 H241. Based on the pr-E protein structure [34], potential key residues in pr-E interaction are indicated by their numbers in the DENV2 NGC proteins. 5B) H244A mutation inhibits pr-E binding in pull-down assay. WT or H244A mutant forms of DI/II were assayed for binding to pr-sepharose beads as in FIG. 2A. 5C) H244A mutation inhibits pr-E binding in SPR assay. WT or H244A mutant forms of DI/II were assayed for binding to pr at various pH values using SPR as in FIG. 2C, shifting to buffer alone at 300 s. Where indicated, mAb 4G2 (molar ratio 1:1) was pre-incubated 15 min at room temperature with DI/II proteins at pH 6.0 prior to assay. Data are a representative example of two independent experiments. SEQ ID NOS. 10-17 for sequences, top to bottom in 5A, respectively.

FIG. 6. H244A E protein interacts with membranes and is resistant to inhibition by pr. WT or H244A DENV2 DI/II proteins (40 μg/ml) were mixed with DIII or BSA (200 μg/ml) in the presence or absence of pr peptide (200 μg/ml). Liposomes were added at a final concentration of 1 mM, and the samples were incubated at pH 5.75 for a total of 60 min at 28° C. Samples were analyzed by floatation on sucrose gradients at pH 5.75 as in FIG. 3A. Data are a representative example of two independent experiments.

FIG. 7. DENV E H244A mutation inhibits virus infection. RNAs derived from the WT and E H244A mutant DENV1 WP infectious clones were electroporated into BHK cells. Cells were cultured for 3 d and infected cells were detected by immunofluorescence. In parallel, cells were cultured at 28° C. for 6 d and progeny virus in the culture medium was quantitated using infectious center assays on indicator BHK cells. Progeny virus titers are shown in the box below each fluorescence image. Results are given for two independent infectious clones of H244A, indicated as (2) and (4). Bar represents 30 μM.

FIG. 8A-8C. DENV E H244A mutation inhibits release of virus-like particles via a low pH-dependent mechanism. 8A) WT and H244A mutant E proteins are comparably expressed. Stable cells inducibly expressing the WT or H244A mutant forms of prM-E were treated with tetracycline for 36 h at 37° C. E protein expression was detected by immunofluorescence and the nuclei were stained with DAPI. Fluorescence images are shown at the same magnification and exposure time. Bar represents 30 μm. 8B) WT and H244A mutant E proteins are comparably immunoprecipitated by conformation-specific mAbs. Stable cells inducibly expressing the WT or H244A mutant forms of prM-E were treated with tetracycline for 36 h at 37° C. E proteins in the cell lysates were immunoprecipitated by Sango, a rabbit polyclonal antibody to DIII, and by the mouse mAbs 4G2 and 4E11, as indicated at the top of the panel. Samples were then analyzed by SDS-PAGE and western blot using mouse anti-DENV2 Ab for the Sango samples and Sango for the mAbs samples. Asterisks indicate the positions of the IgG and IgG heavy chain, which cross-react in the western blot. Equivalent sample input was evaluated by western blot for β-actin (lower panel). 8C) Effect of low pH on WT and H244A VLP production. WT and H244A mutant cells were incubated with tetracycline for 2 h and then in this medium plus 20 mM NH4Cl where indicated for a total of 36 h. VLP released in the culture media were pelleted by ultracentrifugation, and E proteins in the cell lysates were immunoprecipitated using mAb 4G2. VLP and lysate samples were analyzed by SDS-PAGE and western blot using Sango. 5-fold more culture media from the H244A cells than the WT cells were loaded. Data are representative examples of two or more independent experiments.

### DETAILED DESCRIPTION OF THE INVENTION

A method is provided for treating or preventing infection by a flavivirus of a cell in a subject comprising administering to the subject an amount of an agent effective to (i) inhibit interaction of a pr peptide of the flavivirus and a membrane fusion protein E (E protein) of the flavivirus, or (ii) inhibit interaction between the E protein of the flavivirus and the membrane of the cell.

A method is provided for determining if an agent can prevent or reduce exocytosis from a flavivirus-infected cell of a flavivirus virus synthesized by the cell comprising:
a) contacting a membrane fusion protein E (“E protein”), or portion thereof, of the flavivirus with (i) a flavivirus pr peptide, and (ii) the agent under conditions permitting the pr peptide to bind to the E protein or portion thereof; and
b) quantifying the binding between the pr peptide and the E protein or portion thereof, wherein a decrease in the binding between the pr peptide and the E protein in the presence of the agent relative to binding between the pr peptide and the E protein in the absence of the agent indicates that the agent can prevent or reduce exocytosis of the flavivirus virus synthesized by the cell from the flavivirus-infected cell, while no change in or an increase in the binding between the pr peptide and the E protein in the presence of the agent relative to binding between the pr peptide and the E protein in the absence of the agent indicates that the agent is not useful to prevent or reduce exocytosis of the flavivirus virus from the flavivirus-infected cell.

A method is provided for determining if an agent can reduce or prevent fusion of a flavivirus with a cell membrane comprising:
a) contacting a membrane fusion protein E (“E protein”), or portion thereof, of the flavivirus with a flavivirus pr peptide and the agent under conditions permitting the pr peptide to bind to the E protein or portion thereof; and
b) quantifying the binding between the pr peptide and the E protein or portion thereof, wherein an increase in the binding between the pr peptide and the E protein in the presence of the agent relative to binding of the pr peptide and the E protein in the absence of the agent indicates that the agent can reduce or prevent fusion of the flavivirus with the cell membrane, while no change in or a decrease in the binding between the pr peptide and the E protein in the presence of the agent relative to binding between the pr peptide and the E protein in the absence of the agent indicates that the agent is not useful to reduce or prevent fusion of flavivirus with a cell membrane.

In an embodiment the agent is membrane-permeable. In an embodiment the agent is membrane-impermeable. In an embodiment the agent is an organic molecule having a molecular weight of 800 daltons or less. In an embodiment the portion of the E protein is contacted with the flavivirus pr peptide and the agent, and wherein the portion of the E protein comprises E protein domain I and E protein domain II. In an embodiment the portion of the E protein is derived from a Dengue virus.

In an embodiment the Dengue virus is a Dengue Virus 2. In an embodiment the Dengue Virus 2 is a Dengue Virus 2 New Guinea C strain. In an embodiment the flavivirus is a Dengue virus.

In an embodiment the agent effective to inhibit interaction between the E protein of the flavivirus and the membrane of the cell is an isolated pr peptide or an isolated fragment of a pr peptide.

In an embodiment the isolated pr peptide or the isolated fragment of a pr peptide is derived from a Dengue virus. In an embodiment the isolated pr peptide or the isolated fragment of a pr peptide is derived from a Dengue virus New Guinea C strain. In an embodiment the pr peptide is fluorescently tagged.

In an embodiment the agent comprises residues 1-86 of the prM-E protein of Dengue Virus 2 New Guinea C strain. In it comprises residues 1-86 of SEQ ID NO:1.

In an embodiment the agent comprises residues 1-91 of the prM-E protein of Dengue Virus 2 New Guinea C strain. In it comprises residues 1-91 of SEQ ID NO:1.

In an embodiment the portion of the E protein is contacted with the flavivirus pr peptide, and the portion of the E-protein is a soluble truncated E-protein or a Dengue virus 2 ij loop having the sequence set forth in SEQ ID NO:4. In an embodiment the conditions permitting the pr peptide to bind to the E protein or portion thereof comprise an acidic pH.

In an embodiment the methods further comprise, when an agent is found to increase the binding of the pr peptide to the E protein, determining if the agent increases the binding of the pr peptide to the E protein at a neutral pH.

In an embodiment quantifying the binding between the pr peptide and the E protein or portion thereof comprises using fluorescence polarization, surface plasmon resonance, and/or a pull-down assay.

In an embodiment the E protein or portion thereof is contacted with the pr peptide and the agent at approximately the same time. In an embodiment the E protein or portion thereof is contacted with the pr peptide and incubated therewith prior to contacting with the agent. In an embodiment the E protein or portion thereof is incubated with the pr peptide for between 2 minutes and 2 hours

In an embodiment the E protein is in the form of a dimer or a trimer.

A peptide is provided comprising consecutive amino acid residues having the sequence TFKNPHAKKQDVVV (SEQ ID NO:4). In an embodiment the peptide is GCTFKNPHAKKQDVVVC (SEQ ID NO:5).

A pharmaceutical composition is provided comprising the peptide of claim 20 or 21 and a pharmaceutically acceptable carrier.

A peptide is provided comprising consecutive amino acid residues having the sequence TFKNPHAKKQDVVV (SEQ ID NO:4) for treating or preventing infection of a cell in a subject by a flavivirus. In an embodiment peptide is for treating or preventing infection of a cell in a subject by Dengue virus. As used herein a “primary infection” is when a virus infects a first cell, e.g. in a subject. A secondary infection is when virus, created inside of and released from the first cell, infects additional cells. A viral infection may occur in vivo or in vitro. To “treat” an infection by an alphavirus or Flavivirus means to stabilize the infection, to reduce one or more symptoms thereof, and/or to reduce the level of infection (e.g. as determined by viral copy number or viral load in the subject). To “prevent” an infection by an alphavirus or Flavivirus means to stop primary infection of a subject (i.e. of a cell in the subject) or to prevent secondary infection of cells in the subject after a primary infection has occurred.

Agents can be administered in any fashion known in the art for anti-virals including, but not limited to injection, nasal administration, by aerosol, and oral administration. Any acceptable route of administration of the active compounds described herein can be used. For example, oral, lingual, sublingual, buccal, parenteral, intrabuccal, intrathecal, intracerebroventricular, or nasal administration can be effected without undue experimentation by means well known in the art.

For the purpose of oral therapeutic administration, the pharmaceutical compositions of the present invention may be incorporated with excipients. Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

For nasal administration, including for administration via the olfactory epithelia, the active compound or a composition comprising such is administered to the mucous membranes of the nasal passage or nasal cavity of the patient. Pharmaceutical compositions for nasal administration include compositions prepared by well-known methods to be administered, for example, as a nasal spray, nasal drop, suspension, gel, ointment, cream or powder. Administration of the active compound or a composition comprising such may also take place using a nasal tampon or nasal sponge.

For administration parenterally, such as, for example, by intravenous, intramuscular, intrathecal or subcutaneous injection, administration can be accomplished by incorporating the active compound or a composition comprising such of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials

The agent may be associated with a pharmaceutically-acceptable carrier, thereby comprising a pharmaceutical composition. The pharmaceutical composition may comprise the agent in a pharmaceutically acceptable carrier. Alternatively, the pharmaceutical composition may consist essentially of the agent in a pharmaceutically acceptable carrier. Yet alternatively, the pharmaceutical composition may consist of the agent in a pharmaceutically acceptable carrier.

The pharmaceutically-acceptable carrier must be compatible with the agent, and not unduly deleterious to the subject. Examples of acceptable pharmaceutical carriers include carboxymethylcellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage and may be prepared by any method known in the pharmaceutical art. For example, the agent may be brought into association with a carrier or diluent, as a suspension or solution. Optionally, one or more accessory ingredients, such as buffers, flavoring agents, surface active ingredients, and the like, may also be added. The choice of carriers will depend on the method of administration. The pharmaceutical composition would be useful for administering the agent to a subject to prevent or treat alphavirus or flavivirus infection. The agent is provided in amounts effective to prevent or treat alphavirus or flavivirus infection in the subject. These amounts may be readily determined by one in the art. In one embodiment, the agent is the sole active pharmaceutical ingredient in the formulation or composition. In another embodiment, there may be a number of active pharmaceutical ingredients in the formulation or composition aside from the putative agent. In this embodiment, the other active pharmaceutical ingredients in the formulation or composition must be compatible with the agent.

Flavivirus membrane fusion protein E (E protein) is necessary for both primary and secondary infection. E protein has a complicated maturation. E proteins are assembled as immature heterodimers of E protein and precursor membrane protein (prM). After furin cleavage in the low-pH environment of the trans-Golgi network, the pr peptide remains on the E protein dimer, inhibiting the dimer from low-pH fusion with the membrane. The pr peptide dissociates from the E protein dimer in the neutral extracellular space, thus allowing the virus to become low pH-responsive and infectious. In the low-pH environment of the endosome, the E protein dimer dissociates and forms a heterotrimer which mediates fusion with the endosome membrane and virus infection. The E protein ectodomain contains three regions, domains I, II, and III. The pr peptide binds to the E protein and to a portion of the E protein composed of domains I and II (DI/DII). Domains I and II of the E protein of dengue virus 2 comprise residues 1 to 291. Domains I and II of the E protein of other flaviviruses can be determined by one of skill in the art.

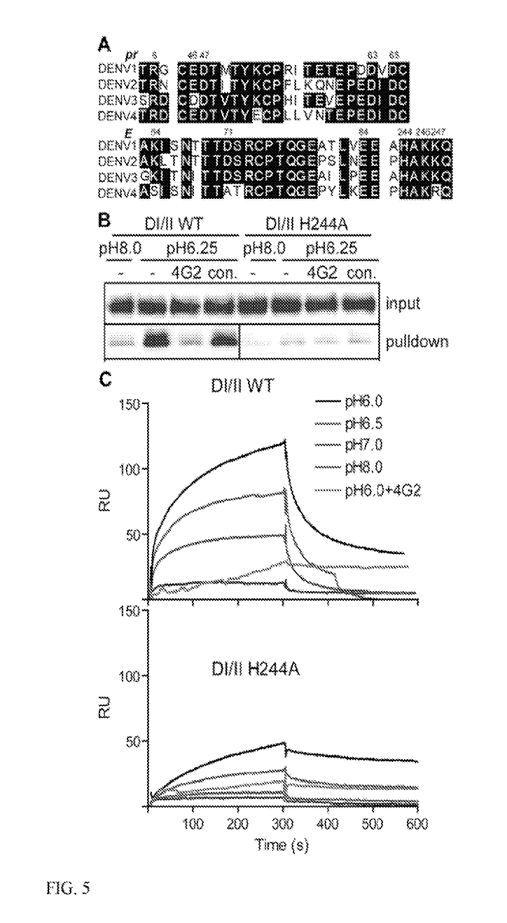

The binding interface between prM and E contains three complementary electrostatic patches containing 11 residues. Sequence analysis shows that these 11 residues are highly conserved among the 4 DENV serotypes (FIG. 5A). The E protein sequence of Dengue virus 2 is mrcigisnrd fvegvsggsw vdivlehgsc vttmaknkpt ldfeliktea kqpatlrkyc ieakltnttt dsrcptqgep slneeqdkrf vckhsmvdrg wgngcglfgk ggivtcamft ckknmkgkvv qpenleytiv itphsgeeha vgndtgkhgk eikitpqssi teaeltgygt vtmecsprtg ldfnemvllq menkawlvhr qwfldlplpw 1pgadtqgsn wiqketivtf knphakkqdv vvIgsqegam htaltgatei qmssgnllft ghlkcrlrmdk 1q1kgmsys mctgkfkvvk eiaetqhgti virvqyegdg spckipfeim dlekrhvlgr litvnpivte kdspvnieae ppfgdsyiii gvepgq1kIn wfkkgssigq miettmrgak rmailgdtaw dfgslggvft sigkalhqvf gaiygaafsg vswtmkilig viitwigmns rstslsyslv lvgvvtlylg vmvqa. (SEQ ID NO:1) Histidine residue 244 of the E protein is conserved across flaviviruses. The sequence for domain I and domain II of dengue virus 2 is mrcigisnrd fvegvsggsw vdivlehgsc vttmaknkpt ldfeliktea kqpatlrkyc ieakltnttt dsrcptqgep slneeqdkrf vckhsmvdrg wgngcglfgk ggivtcamft ckknmkgkvv qpenleytiv itphsgeeha vgndtgkhgk eikitpqssi teaeltgygt vtmecsprtg ldfnemvllq menkawlvhr qwfldlplpw lpgadtqgsn wiqketivtf knphakkqdv vvIgsqegam htaltgatei qmssgnllft ghlkcrlrmdk 1 (SEQ ID NO:2). The pr peptide sequence of dengue virus 2 is fhlttrngep hmivsmekg ksllfktedg vnmctlmamd lgelcedtit ykcpflrqne pedidcwcns tstwvtygtc tttgehrrek r. (SEQ ID NO:3)

An agent may decrease the binding between the E protein or portion thereof and the pr peptide in a number of ways, including but not limited to, binding to the E protein or portion thereof in the same location as the pr peptide would bind to the E protein or portion thereof; binding to the E protein or portion thereof at a remote site and changing the conformation of the E protein or portion thereof at the site of pr peptide binding, thereby inhibiting pr peptide binding with the E protein or portion thereof; binding to the pr peptide at the same location as the pr peptide would bind to the E protein or portion thereof; or binding to the pr peptide at a remote site and changing the conformation of the pr peptide at the site the E protein or portion thereof would bind, thereby inhibiting pr peptide binding with the E protein or portion thereof. The agent may inhibit binding between E protein or portion thereof and pr peptide at low pH, such as that existing in a trans-Golgi network, or at neutral pH, such as that experienced in the endoplasmic reticulum (ER) or extracellularly, and/or may bind the E protein or portion thereof at low pH and/or neutral pH. An agent that inhibits binding between E protein or portion thereof and pr peptide or that interacts with E protein or portion thereof at low pH may prevent secondary infection by causing the E protein to permanently fuse with the membrane in the primary infection cell, or by causing misfolding of the E protein during biosynthesis. An agent that affects pr peptide binding to E protein or portion thereof at both low and neutral pH may prevent secondary infection by inhibiting E protein from fusing to the endosome of the secondary cell.

The agent in the present invention can be any chemical or biological agent for example, a small molecule (i.e. an organic molecule of 800 daltons or less), an antibody or fragment thereof, a peptide, a polypeptide, protein, a protein aptamer, a protein fragment, peptide mimetic, or an aptamer. In an embodiment, the agent is biomembrane-permeable. In an embodiment, the agent is biomembrane-impermeable. An aptamer is a single stranded oligonucleotide or oligonucleotide analog that binds to a particular target molecule, such as a protein. A protein aptamer is a variable peptide loop attached at both ends to a protein scaffold that interferes with protein interaction. A peptide mimetic is a short peptide which mimics the sequence of a protein of interest.

Interaction or binding between E protein or portion thereof and the pr peptide in the presence of the putative agent or the interaction between E protein or portion thereof and the agent can be measured by any method known in the art including, but not limited to, pull-down assay, surface plasmon resonance, and fluorescence polarization. For fluorescence polarization, the pr peptide or the agent may be fluorescently tagged. The motion of the tagged protein, peptide, or agent can be viewed with fluorescence polarization. If a tagged particle interacts with a larger protein, peptide, agent, or other molecule, the motion of the tagged particle will change. A pull-down assay is an in vitro affinity method for determining physical interaction between two or more proteins or peptides. A purified and tagged protein or peptide is immobilized and proteins that interact with this first tagged protein or peptide are “pulled-down” and immobilized. When a surface is coupled with a biopolymer, such as a protein or peptide, adsorption of molecules onto that biopolymer can be measured with surface plasmon resonance.

The interaction or binding between E protein or a portion thereof and pr peptide in the presence of the agent, or the interaction between E protein or portion thereof and the agent can be compared to the interaction or binding in a control. When measuring the interaction between E protein or portion thereof and pr peptide in the presence of the agent, the control may be, but is not limited to: measuring the interaction between E protein or portion thereof and pr peptide without the agent; contacting E protein or portion thereof having a mutated histidine residue no. 244 with pr peptide and the agent, and measuring the interaction between the pr peptide and the E protein or portion thereof having a mutated histidine residue no. 244; or contacting alphavirus fusion protein E1 or a portion thereof with pr protein and the agent and measuring the interaction between the alphavirus fusion protein E1 or portion thereof and pr peptide. Mutation of histidine residue 244 (H244) of the E protein or portion thereof, e.g. of a flavivirus, by replacing H244 with another amino acid, such as alanine (H244A), inhibits the interaction between the E protein or portion thereof having a mutated histidine residue no. 244 and pr peptide. E protein or portion thereof may be mutated in any way known in the art. Preferably, histidine residue no. 244 of the E protein or portion thereof is replaced with alanine. Alphavirus class II fusion proteins are structurally related to the E protein but do not interact with pr peptide. When measuring the interaction between E protein or portion thereof and the agent, the control may be, but is not limited to: contacting E protein or portion thereof having mutated histidine residue 244 with the agent, and measuring the interaction between the agent and the E protein or portion thereof having mutated histidine residue 244; or contacting alphavirus fusion protein E1 or a portion thereof with the agent and measuring the interaction between the alphavirus fusion protein E1 or portion thereof and the agent.

In an embodiment, the pH is between 6 and 8. In order to mimic the low-pH environment experienced by the E protein and pr peptide, the pH is more preferably between 6 and 6.5. When mimicking the low-pH environment experienced by E protein and the pr peptide, most preferably the pH is 6.25. The pH may also be neutral in order to mimic the extracellular pH or the ER pH.

The pr peptide may be added to the E protein or portion thereof at the same time as the agent. Alternatively, the pr peptide may be added to the E protein or portion thereof and incubated, preferably between 2 minutes and 2 hours, before the agent is added. In such a case, the interaction or binding between the pr peptide and the E protein or portion thereof will be disrupted only if the agent's interaction with the E protein or portion thereof or the pr peptide is much more favorable.

A viral infection may be prevented by administering the agent before a subject has encountered the virus. A viral infection may be treated by administering the agent after a subject has been infected with a virus.

The present invention further provides a kit for screening an agent that prevents or treats viral infection by inhibiting the interaction between a pr peptide and a membrane fusion protein E (E protein) or a portion thereof, or by interaction with the E protein or portion thereof, the kit comprising E protein or portion thereof in a medium, wherein the portion of the E protein comprises domain I and domain II

The present invention may be performed with high throughput arrays, such as a 384-well plate format. Any assay known in the art may be used, including but not limited to, binding assays such as immunospecific assays, affinity assays, or fluorescence-based assays.

The medium may be any appropriate medium known in the art. Preferably, the medium has a pH between 6 and 8. In order to mimic the low-pH environment experienced by the E protein and pr peptide, the pH of the medium is more preferably between 6 and 6.5. When mimicking the low-pH environment experienced by E protein and the pr peptide, most preferably the pH of the medium is 6.25. The pH of the medium may also be neutral in order to mimic the extracellular pH or ER pH. The medium is preferably a physiological medium, or one which mimics the extracellular fluid or interstitial fluid.

The assay or kit may include at least one control. A control may be, but is not limited to: medium; E protein or portion thereof with mutated histidine residue 244 in a medium; alphavirus fusion protein in a medium. Mutation of the E protein histidine residue 244 to another amino acid, such as to an alanine, inhibits the interaction between E protein or portion thereof and pr peptide.

Herein a system is disclosed to produce DENV pr peptide and reconstitute the pr-E interaction in vitro. At low pH pr bound to both monomeric and dimeric forms of E and blocked their membrane insertion and trimerization. Addition of exogenous pr to mature DENV particles inhibited virus fusion and infection. Mutation of a key histidine residue in the pr-E interface, E H244, reduced pr's binding and inhibitory activity, and reduced DENV secondary infection and particle production. The defect in particle production could be partially rescued by neutralization of exocytic low pH, indicating the important role of pr in protecting DENV from premature fusion during transport to the plasma membrane.

### Experimental Details

Expression and Characterization of pr Peptide.

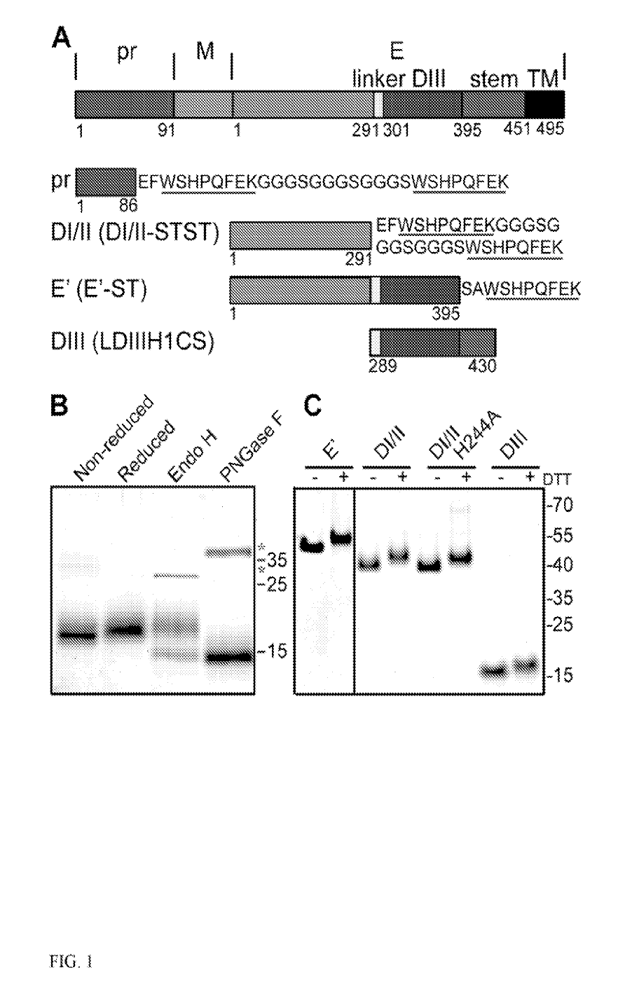

A number of truncated E proteins have been successfully produced by co-expression with prM [e.g., references 30,39], while the pr-E structural studies were based on a secreted hybrid protein containing truncated prM linked to truncated E [34]. Previous studies indicated that full-length TBEV prM could fold correctly when expressed in the absence of E protein [37], suggesting that production of pr peptide alone might be possible. A construct was generated based on residues 1-86 of DENV2 prM, truncating pr just before the start of the furin cleavage recognition site at residue 87 (FIG. 1A). This sequence was linked to a mammalian signal peptide at the N-terminus and to an affinity tag at the C-terminus, and expressed in 293T cells. The protein was isolated in a highly purified form by affinity chromatography and gel filtration (FIG. 1B), and was recognized by mAb prM-6.1 against prM [40] (data not shown). The pr peptide migrated at a position of ˜17 kDa in reducing SDS-PAGE, in keeping with its predicted size of 13 kDa plus the presence of carbohydrate due to the glycosylation site at position 69. This carbohydrate was removed by Peptide N-glycosidase F (PNGase F) to give a peptide of the predicted size. The protein was largely resistant to Endoglyosidase H (Endo H) digestion, indicating maturation of the carbohydrate chain as the protein transited through the Golgi complex. A mobility shift was observed upon reduction of pr, in keeping with the presence of 3 disulfide bonds in the structure of pr [34].

A dimeric ectodomain form of DENV2 E protein was produced and purified containing all three domains (E′), a monomeric form containing E domains I and II (DI/II), and E domain III (DIII) (FIGS. 1A and 1C), all as previously described in detail [30,41].

pH-Dependent Binding of pr and E Proteins.

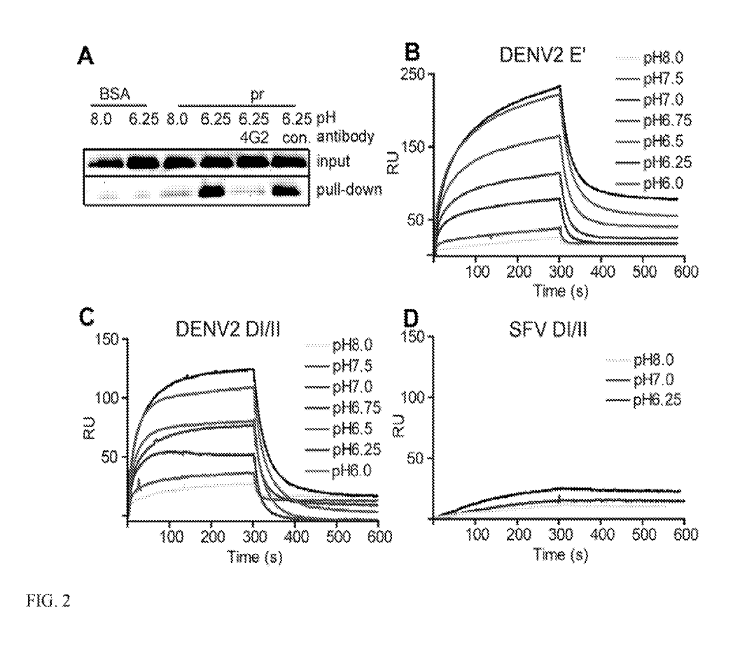

As a first test of in vitro pr-E binding, pr was coupled to sepharose beads and its ability to pull-down truncated E protein containing only domains I and II was tested. This form of E protein is monomeric and the tip of DII is thus accessible even at neutral pH. Previous studies showed that this and other DENV DI/II proteins are active in membrane insertion and trimerization at both neutral and low pH [30]. Efficient pull-down of DI/II protein by pr-sepharose (FIG. 2A) was observed, but in spite of the accessibility of the pr binding site on DI/II at neutral pH, pull-down was low pH-dependent. The pull-down of DI/II protein by pr was specific, as it was blocked by inclusion of mAb 4G2 against the E fusion loop at the DII tip, and did not occur with BSA-sepharose beads. These data suggested that the recombinant pr peptide could bind to the tip of DI/II in a low pH-dependent reaction.

For more detailed studies of pr-E binding, surface plasmon resonance (SPR) assays were performed using our various forms of recombinant E protein with immobilized pr peptide. Compared to the pull-down assay, SPR can detect low levels of protein-protein interactions as binding is detected in real time and does not require removal of unbound E. The E′ protein is a dimer at neutral pH and dissociates to monomers at low pH [30]. When SPR was performed with E′ protein buffered at pH 8.0 there was very low binding (low signal response) (FIG. 2B). As the buffer pH was decreased, the signal gradually increased, with maximal response observed at ˜pH 6.25 and no further increase at pH 6.0. A rapid decrease in signal was observed when the samples were shifted to protein-free buffer, indicating rapid dissociation of the pr-E interaction. Similar results were obtained using monomeric DI/II, with the lowest binding at pH 8.0, highest binding at pH 6.25, and a slight decrease at pH 6.0 (FIG. 2C). Thus, the dimeric E′ and monomeric E DI/II proteins bound pr peptide with similar pH-dependence. Binding to pr was specific, as little interaction was observed using the structurally similar E1 DI/II protein of SFV (FIG. 2D). In addition, binding of DENV E DI/II protein to pr was inhibited by preincubation with mAb 4G2 against the fusion loop (molar ratio 1:1) (data not shown). Determination of the affinity of pr-E binding was not performed as the data did not fit to a simple Langmuir model of 1:1 binding, presumably because of E protein aggregation at low pH.

Effect of Exogenous pr Peptide on E Protein-Membrane Interaction.

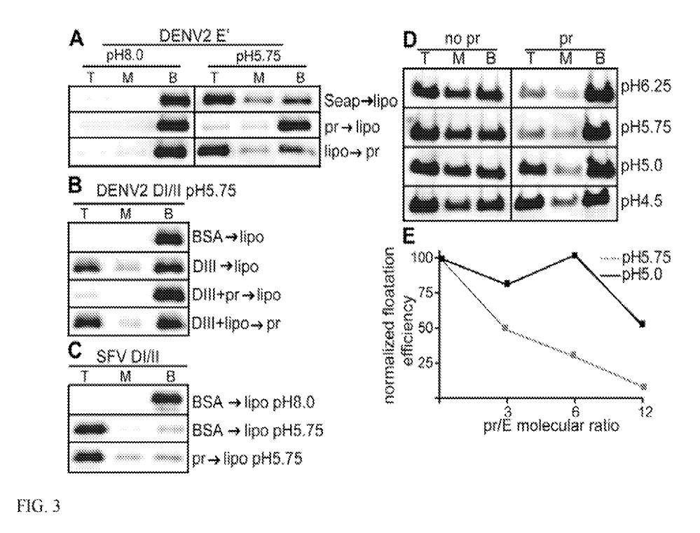

Previous studies showed that retention of endogenous pr peptide on the furin-processed DENV particle inhibits virus interaction with liposomes at low pH [35]. Structural considerations suggested that this inhibition occurs primarily by blocking low pH-triggered dissociation of the E dimer, a required first step in the fusion reaction. To test this mechanism, the effect of pr on the membrane interactions of dimeric and monomeric forms of E protein was evaluated. The E′ dimer was pre-incubated with pr peptide or an unrelated protein with the same affinity tag for 5 min at pH 8.0, and then treated at pH 5.75 in the presence of target liposomes. Membrane-associated proteins were separated by liposome floatation on sucrose gradients. There was no liposome co-floatation when E′ protein was incubated with liposomes at neutral pH (FIG. 3A). About 70% of the total E′ floated with liposomes in the top part of the sucrose gradient after treatment at pH 5.75 in the presence (FIG. 3A, top panel) or absence (data not shown) of a control protein. In contrast, when E′ was preincubated with pr peptide (pr:E′ molar ratio 12:1) and treated with low pH, only ˜2% of E′-ST floated with the liposomes (FIG. 3A, middle panel). Inhibition by pr was not observed when it was added after E′ was treated at low pH in the presence of liposomes for 30 min (FIG. 3A, bottom panel), and thus pr needed to be present during the membrane insertion step. Inhibition was concentration-dependent, with 22% E′ co-floatation at a pr:E′ molar ratio of 3:1, 8% at 6:1, and 0.4% for 24:1 (data not shown; see also FIG. 3E).

The effect of pr on the DENV E DI/II protein was tested. This protein is monomeric and its stable membrane interaction requires DIII to “clamp” the core trimer [30]. As shown in FIG. 3B, ˜25% of DI/II co-floated with liposomes at low pH in the present of DIII, while no co-floatation was detected when BSA was substituted for DIII protein. The addition of pr peptide blocked membrane interaction of DI/II when added prior to liposome incubation (FIG. 3B, 3rd panel), but not after liposome incubation (FIG. 3B, bottom panel).

The structurally related alphavirus protein SFV E1 DI/II is monomeric and efficiently interacts with membranes at low pH (80% cofloatation, FIG. 3C, middle panel). No inhibition occurred when pr peptide was added prior to liposome addition (FIG. 3C, bottom panel), in keeping with the lack of pr-SFV DI/II binding in the SPR experiments discussed above. Thus, pr peptide specifically inhibits target membrane interaction of both monomeric and dimeric forms of the DENV E protein.

E′ protein efficiently inserted into membranes over a wide range of pH values from 6.25-4.5 (FIG. 3D-E). However, pr's inhibition of E membrane insertion was less efficient in the pH range (pH 5.0) present in the late endocytic pathway (FIG. 3D-E). This loss of pr inhibition at more acidic pH may be relevant to recent studies of infection by immature DENV [42], as mentioned in the discussion section below.

Effect of Exogenous pr Peptide on Dengue Virus Fusion and Infection.

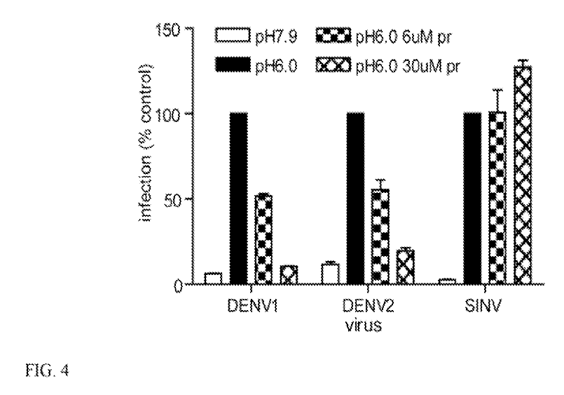

All of the results above were obtained with soluble forms of the E protein. In order to test the ability of exogenous pr peptide to interact with and inhibit intact DENV, a previously described assay was used that monitors low pH-triggered fusion of DENV with cells [41]. In this fusion-infection assay, virus is pre-bound to target cells on ice, and then treated at 37° C. for 1 min at low pH to trigger virus fusion with the plasma membrane. This fusion reaction is then quantitated by detecting the infected cells by immunofluorescence. The effect of pr peptide during this 1 min low pH treatment was tested using DENV1 WP and DENV2 NGC. The sequence of E DI/II is 68% identical between these two serotypes. Both serotypes showed efficient fusion and infection after treatment at pH 6.0, with about a 10-fold increase compared to samples treated at pH 7.9 (FIG. 4). The addition of pr peptide during the 1 min low pH treatment strongly inhibited DENV fusion and infection. Inhibition was dose-dependent, with 45-49% inhibition at 6 μM pr and 81-85-% inhibition at 30 μM pr. In contrast, pr did not inhibit low pH-triggered fusion by the alphavirus SIN (FIG. 4). Thus, exogenous DENV2 pr peptide can specifically interact with mature DENV1 and DENV2 to block virus fusion and infection. Inhibition was not observed when DENV was pre-incubated with 30 μM pr at pH 7.0 and then added to target cells in a standard infection assay, suggesting that under these conditions an inhibitory concentration of pr was not present during low pH-triggered fusion reaction in the endosome. This result also indicates that the presence of pr did not affect virus-cell binding.

Role of E H244 in pr-E Binding.

Although the interaction of pr with DENV can clearly prevent virus-membrane interaction and fusion [this study and 35], the importance of pr in protecting DENV during exocytic transport has not been defined. The binding interface between prM and E contains three complementary electrostatic patches containing 11 residues [34] (see also FIG. S1). Sequence analysis shows that these 11 residues (FIG. 5A, numbered residues) are highly conserved among the 4 DENV serotypes, and that D63 and D65 of pr, and the complementary H244 on E protein are conserved among all reported flavivirus sequences [34]. Optimal pr-E binding in vitro occurred at ˜pH 6.25 (FIG. 2), suggesting that protonation of H244 could be involved in this pH-dependence. To test this alanine was substituted for H244 in the DI/II protein. DI/II H244A was produced in highly purified form with electrophoretic mobility similar to that of the wild type (WT) protein in reducing and non-reducing SDS-PAGE (FIG. 1C).

The effect of the H244A mutation was tested on pr-E binding. In agreement with our earlier results, WT DI/II protein was efficiently pulled-down by pr-sepharose (FIG. 5B). Pull-down was low pH-dependent and blocked by mAb 4G2 against the E fusion loop at the DII tip. In contrast, almost no H244A DI/II protein was pulled-down by pr-sepharose at either low pH or neural pH (FIG. 5B). SPR analysis of WT DI/II protein showed most efficient binding at pH 6.0, and binding was blocked by pre-incubating the DI/II protein with mAb 4G2 (molar ratio 1:1) before dilution into SPR buffer (FIG. 5C, upper panel). Equivalent concentrations of H244A DI/II protein showed greatly reduced binding to pr compared to that of WT protein (FIG. 5C, lower panel). Although H244A binding was decreased, the residual binding was still blocked by mAb 4G2 and had an acidic pH optimum. This suggests that binding also involves other residues in the pr-E interface, such as the complementary residues identified in the structural studies and shown in FIG. 5A.

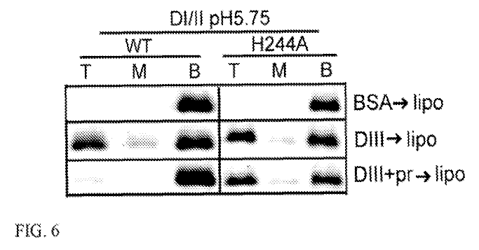

If the H244A DI/II protein was still active in binding to target liposomes was investigated. WT or mutant DI/II proteins were mixed with liposomes at low pH in the presence of DIII protein to stabilize the core trimer. Both proteins efficiently bound liposomes in a DIII-dependent reaction (FIG. 6), indicating that the mutant protein retains its ability to insert into target membranes and form a core trimer. In agreement with the results in FIG. 3C, floatation of the WT protein was blocked by inclusion of pr during the membrane insertion step (FIG. 6). In contrast, the efficiency of floatation of the H244A mutant protein was 43% in the absence of pr and 47% in the presence of pr. Thus, the H244A mutation did not inhibit E-membrane interaction but made that interaction insensitive to the presence of pr.

H244A Mutation Inhibits DENV Secondary Infection.

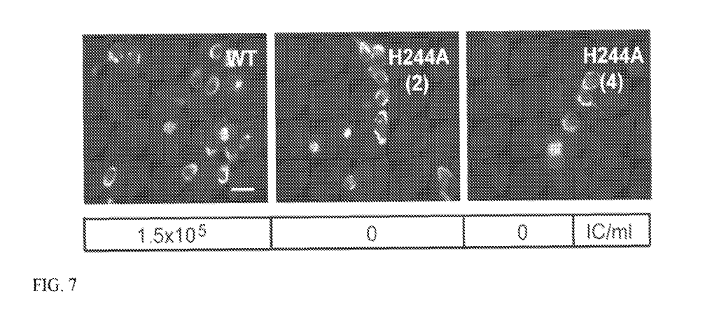

Since the E H244A mutation disrupts E protein's interaction with pr, this mutation was used to address the importance' of pr in protecting DENV during transport through the exocytic pathway. The E H244A mutation was introduced into the infectious clone of DENV1 WP. WT and mutant viral RNAs were prepared by in vitro transcription and were electroporated into BHK cells. After culture for 3 d at 37° C., both WT and mutant RNA-electroporated cells expressed abundant E protein as detected by immunofluorescence microscopy (FIG. 7). Parallel cultures were incubated for 6 d and progeny virus in the culture media was detected by infectious center assays on indicator BHK cells. WT-infected cells produced infectious progeny virus with a titer of ˜1.5×105 IC/ml. However, two independent infectious clones of the H244A mutant produced no detectable progeny virus, even though the viral RNAs mediated efficient primary infection as shown in FIG. 7. This agrees with previous studies indicating lethal effects of an H244A mutation on DENV2 [43].

Role of E H244 During Virus Assembly and Secretion.

The absence of secondary infection by the H244A DENV1 mutant could be due to decreased virus particle production and/or production of particles that are non-infectious. Efficient DENV particle production is dependent on E protein folding, particle budding into the ER, and subsequent particle egress through the secretory pathway. To investigate these issues, the ability of the flavivirus prM and E proteins to assemble into virus-like particles (VLP) in the absence of other viral components or virus infection was taken advantage of [44,45,46]. The VLP system avoids complications arising from selection of revertants of deleterious virus mutations such as H244A. Flavivirus VLP bud into the ER in the immature prM form, undergo furin maturation during transport through the secretory pathway, and display similar low pH-dependent fusion activity as infectious virions [44,47]. The VLP system has been used extensively to follow the process of flavivirus particle production and the role of prM in this process [37,44,45,48].

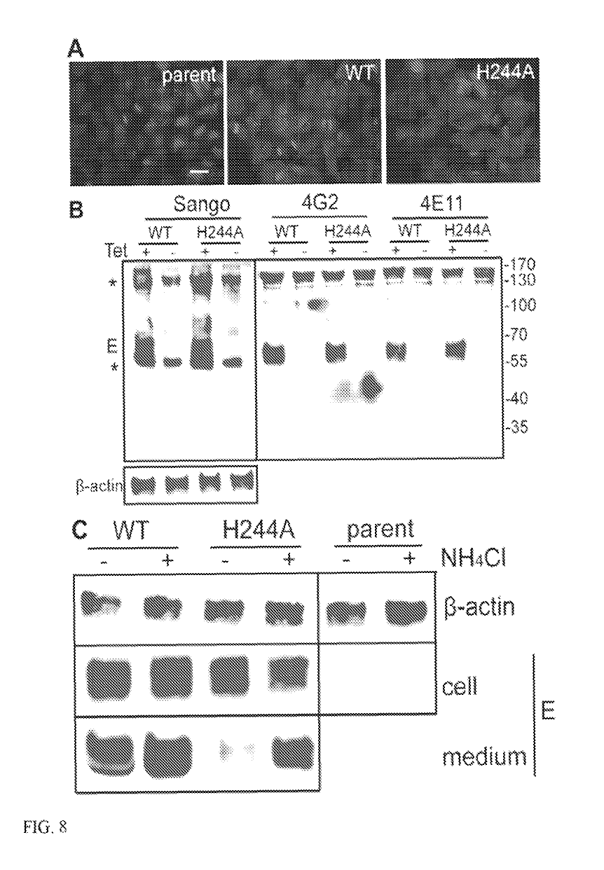

Stable HEK 293 cells were established that inducibly express the DENV1 WT or H244A prM-E proteins. After 36 h induction with tetracycline, both WT and H244A cells show abundant intracellular expression of the DENV1 E protein as detected by immunofluorescence, while the parent cell line is negative for E expression (FIG. 8A). To evaluate whether WT and H244A E proteins were correctly folded, cells were induced for 36 h, lysed, and immunoprecipitated with a rabbit polyclonal antibody to E DIII, and with two conformation-specific mAbs. mAb 4E11 recognizes a discontinuous epitope on DENV E DIII and requires proper DIII disulfide bond formation for recognition [49,50]. mAb 4G2 recognizes the fusion loop at the tip of flavivirus E DII and its epitope is sensitive to reduction [51]. Expression studies have shown that the 4G2 epitope is not formed if the E protein is expressed in the absence of prM [52], indicating that this epitope is particularly useful for diagnostic tests of prM's chaperone interaction with E [see also reference 37]. As shown in FIG. 8B, lysates from cells induced to express prM plus WT or H244A E proteins showed strong reactivity with all three antibodies. Quantitation of multiple experiments confirmed that WT and H244A E proteins were comparably recognized by the 4E11 and 4G2 mAbs. Thus, by these criteria H244A E protein interacts with prM protein and is correctly folded. This result also agrees with the finding that truncated H244A E protein expressed with prM in the S2 cell system was fully active in low pH-dependent membrane binding and trimerization, suggesting correct folding (FIG. 6).

Inducible cells were then used to examine VLP production. Expression was induced for 36 h. The cells were then lysed and the E proteins immunoprecipitated, and the VLP in the culture media were pelleted by ultracentrifugation. Analysis by western blotting showed strong E protein expression in both WT and H244A cells, and no expression in the parent cells (FIG. 8C). The WT cells released E protein in VLP, but VLP release from cells expressing the H244A mutant E protein was greatly reduced (FIG. 8C, −media samples). This result is in keeping with the hypothesis that the H244A cells assemble VLP in the neutral pH environment of the ER but that VLP release is inhibited by the lack of pr protection from the low pH of the secretory pathway. To test this idea, we induced WT and H244A prM-E expression and cultured the cells in the presence of 20 mM NH₄Cl to neutralize the acidic pH in the Golgi and TGN compartments (FIG. 8C, +NH₄Cl lanes). The cellular expression level of either E protein was not significantly affected by NH₄Cl treatment, and WT VLP production was similar in NH₄Cl-treated cells and untreated cells. However, production of VLP containing the H244A mutant E protein was increased 4-7 fold in NH₄Cl-treated cells. While H244A VLP production was still significantly decreased compared to that of WT, it was selectively rescued by NH₄Cl treatment.

The ij Loop.

The ij loop peptide (TFKNPHAKKQDVVV (SEQ ID NO:4) from the Dengue virus E protein is assayed as an agent which binds to pr. The ij loop peptide can be modified to comprise, for example, an N-terminal fluorophore such as fluorescein, a glycine linker, and two cysteines so as to permit cyclization via a disulfide bridge (e.g. GCTFKNPHAKKQDVVVC (SEQ ID NO:5). The carboxy-terminus may be amidated. The binding of the it loop peptide to pr peptide, e.g. of a Dengue virus, can be used in an assay to determine molecules which inhibit the binding the pr peptide to the ij loop peptide (and therefore to the E protein). Such binding can be determined by common techniques such as fluorescence polarization.

Discussion

During translation of the flavivirus polyprotein, prM is the first protein translocated into the ER lumen, where it acts as a chaperone during the folding of the subsequently translocated E protein [4,37,44]. In addition to this important role of prM during E protein synthesis, a variety of data suggest that the interaction of pr peptide with the viral E protein protects flaviviruses from low pH during their transport through the exocytic pathway [34,35,36]. Here it is disclosed that a recombinant pr peptide was efficiently folded, glycosylated, and secreted from 293T cells in the absence of its normal prM context and furin processing. Recombinant pr bound to soluble E proteins at low pH, inhibited E-membrane insertion, and interacted with mature dengue virus to block fusion and infection. Alanine substitution of the conserved E H244 within the pr-E interface disrupted pr-E binding in vitro and blocked secondary virus infection. VLP production was inhibited by the H244A mutation and partially rescued by pH neutralization with NH₄Cl. Together our data demonstrate the critical role of pr in protecting DENV from exocytic low pH.

Properties of pr-E binding: The in vitro interaction of pr with various truncated forms of E protein was strongly pH-dependent, with a pH optimum of ˜6.25. In situ measurements indicate that the pH of the TGN is ˜6 [53], while the pH optimum of DENV2 NGC fusion is ˜6.2 [41]. The low pH of the TGN is critical for the rearrangement of immature DENV to allow furin cleavage, but once the virus is processed it becomes fusion-active in this same pH range. Thus the pH dependence of the pr-E interaction appears optimized to protect DENV during its continued transit through the secretory pathway. Pr's inhibition of E membrane insertion was less efficient at a pH value (pH 5.0) similar to that in the late endocytic pathway (FIG. 3D-E). This loss of pr inhibition at more acidic pH could help to explain the recent finding that infection by immature DENV is enhanced by antibodies to prM [42]. The antibody-bound immature virus is likely to be endocytosed and processed by cellular furin in the endocytic pathway [54]. The lower pH conditions of the late endocytic pathway could then cause the loss of pr inhibition and allow virus fusion.

The structure of furin-cleaved DENV at pH 6.0 shows that pr is bound to the virion through interactions with the DII tip of one E protein and DI on the neighboring E monomer [35,36]. This suggested that pr might primarily block virus-membrane interaction by preventing dissociation of E dimers, a required first step in the fusion pathway [55]. Our results show efficient binding of pr to the dimeric form of the DENV E protein, but also to the monomeric DI/II form. It is not known if the E′ protein dimer is stabilized by pr interaction or if the dimer dissociates prior to interaction with pr, and experiments to address these points were inconclusive (data not shown). The similar pH dependence of pr binding to monomeric and dimeric E proteins suggests that pr may bind the same site in both cases. mAb 4G2 against the fusion loop inhibited pr interaction with E DI/II, confirming that pr was binding to the DII tip rather than to other sites on expressed E proteins. In keeping with its binding site in the vicinity of the fusion loop, pr peptide blocked the membrane insertion and liposome co-floatation of E′ and DI/II proteins. Prior studies showed that a monomeric DI/II protein with a single Strep affinity tag stably inserts into liposomes at either neutral or low pH [30], and pr blocked this insertion even at pH 8.0 where its interaction with DI/II was suboptimal (data not shown). Thus, while the pr-E interaction is strongly low pH-dependent, its functional inhibition of membrane insertion can still be observed at neutral pH in the presence of excess pr.

Effects of E protein H244 mutations: Several other studies have addressed the role of E H244 in the flavivirus lifecycle. Experiments in TBEV evaluated particle production and membrane fusion activity using a VLP system [56]. Mutation of H248 (TBE numbering) to A or I blocks VLP secretion, in agreement with our results. However, an H248N mutant efficiently produces VLP, and these particles show WT levels of fusion activity. WNV E H246A or Q mutations inhibit release of infectious reporter virus particles from cells, as do a number of other substitutions at this position [57]. Replacement of H246 with aromatic residues such as phenylalanine allows both particle release and infectivity. An H244A mutation in DENV2 NGC inhibits infectious virus production [43]. E H244 and its interacting partners D63 and D65 on pr are conserved within the flaviviruses, and thus these data from several flaviviruses plus our DENV results support an important role for the E 244 position. However, a histidine residue at this position does not seem to be strictly required for particle production, suggesting that substitutions such as 244F and 244N can support the interaction of E with pr.

In contrast to the block in production of H244A VLP, the H244A DI/II protein was efficiently secreted from cells. Mutant protein secretion was somewhat reduced, with the final yield of DI/II H244A about half that of the WT protein in two separate preparations (data not shown), suggesting some effects of non-optimal pr interaction. However, unlike the E protein in virus or VLP, the truncated DI/II protein lacks the TM region and does not mediate membrane fusion, and thus may be relatively independent of the pH-protection function of pr. The purified WT and mutant DI/II proteins were able to bind liposomes and form core trimers that were stabilized by DIII (FIG. 6). Thus, the mutant protein is correctly folded and active in membrane insertion. Studies with conformation-specific mAbs also provided evidence for the correct folding of H244A E protein (FIG. 8B). Together, these results suggest that the H244A E protein is still able to access the chaperone functions of prM, while its decreased pr binding indicates that it can no longer utilize the pH protection functions of pr.

These data are consistent with the idea that, similar to WT E, the mutant protein is assembled with prM into VLP in the ER. The membrane insertion and trimerization activity of H244A suggest that the full-length mutant protein would be fusion-active on such VLP once they are transported from the neutral pH of the ER to the low pH of the Golgi and TGN [31]. Thus, the decreased release of H244A VLP and its partial rescue by neutralization of the exocytic pathway support a critical role for pr in protecting DENV from exocytic low pH, and suggest that virus/VLP fuses in the TGN in the absence of pr-E interaction. Rescue of H244A VLP production by NH4Cl was clearly incomplete. This may be due to complex aspects of both virus and cell, such as direct effects of the H244A mutation on particle assembly in the ER, or difficulties in blocking fusion of a virus with the relatively high pH threshold of DENV.

Implications of the in vitro pr-E interaction: Several strategies have been used to block flavivirus and alphavirus fusion reactions and thus inhibit virus infection. SFV and DENV fusion are specifically blocked by exogenous DIII, which binds to the core trimer and prevents the foldback of endogenous DIII and hairpin formation [41]. A later stage in DENV fusion is targeted by a stem-derived peptide, which binds to the ectodomain trimer in which DIII has folded back but stem packing has not yet occurred [58]. These virus protein-protein interactions can be reconstituted in vitro [29,30,58], permitting their use as screens for small molecule inhibitors of virus fusion and infection.

The in vitro reconstitution of the pr-E interaction using soluble components is a screen for small molecule inhibitors of this important flavivirus protein-protein interaction. Such inhibitors could act at multiple points in the virus lifecycle. During virus protein biosynthesis, an inhibitor could block the chaperone interaction of prM with E, leading to misfolding of E and its elimination by the ER quality control pathway. An inhibitor of pr interaction could make E protein susceptible to premature fusion in the TGN and could thus block virus production similar to the H244A mutation. It is also possible that small molecule inhibitors of pr-E binding could interact directly with the DII tip on mature virus particles, perhaps stabilizing the dimer and/or blocking membrane insertion of the fusion loop, thereby blocking virus fusion. Thus the in vitro system described here has the potential to identify molecules that could aid in the study of the flavivirus lifecycle and that could act to inhibit specific steps.

Effects of pr on virus fusion: Previous studies showed that after cleavage endogenous pr is retained on the virus particle if the virus is maintained at acidic pH [35]. Under these conditions, the virus-pr complex does not bind target membranes, while virus from which pr is first released at neutral pH efficiently binds membranes upon shift to acid pH. Thus, the bound endogenous pr inhibits virus-membrane interaction and presumably blocks virus fusion [35]. The results demonstrated that even after maturation to fully infectious DENV particles, exogenous pr could add back to the virus and inhibit low pH-triggered virus fusion and infection. The flavivirus membrane fusion reaction is very rapid, occurring within seconds of low pH treatment [47]. Recombinant DENV2 pr peptide inhibited fusion by both DENV1 and DENV2, suggestive of a fairly broad spectrum inhibition in agreement with the strong sequence conservation at the pr-E interface [34].

The structure of the flavivirus E protein in its pre-fusion and post-fusion conformations defines the dramatic conformational changes between these two states. Many questions about the intermediates that connect the pre- and post-fusion conformations remain. In particular, it will be important to define the membrane protein rearrangements in the context of the highly organized flavivirus particle. For example, a neutralizing E mAb that blocks virus fusion was used to trap a West Nile virus fusion intermediate [59]. It will be interesting to evaluate if exogenous pr peptide could also be used as a novel probe to capture intermediates in the flavivirus fusion pathway.

### Materials and Methods.

Cells, viruses and antibodies: BHK-21 cells and C6/36 mosquito cells were cultured as described previously [60]. 293T cells and T-REx™-293 cells (Invitrogen, now Life Technologies, Carlsbad, Calif.) were cultured as previously described using tetracycline-deficient fetal calf serum for the latter cells [61]. The DENV2 New Guinea C (NGC) strain and the DENV1 Western Pacific (WP) strain were propagated in C6/36 cells in DMEM containing 2% heat-inactivated fetal calf serum and 10 mM Hepes, pH 8.0, as previously described [41,62]. The DENV2 New Guinea C (NGC) strain is available from the ATCC, Manassas, Va., USA under ATCC® Number: VR-1584. See also K. Irie et al. (1989) [71]. Sequence analysis of cloned dengue virus type 2 genome (New Guinea-C strain). Sindbis virus expressing green fluorescent protein was obtained as an infectious clone (a kind gift from Dr. Hans Heidner) and propagated in BHK cells [63].

4G2 is a mouse monoclonal antibody (mAb) that recognizes the fusion loop of flavivirus E proteins [51,64]. mAb prM-6.1 recognizes a linear epitope on prM, and was a kind gift of Drs. Chunya Puttikhunt and Nopporn Sittisombut [40]. 4E11 is a mouse mAb that recognizes DIII of DENV E protein and neutralizes all 4 serotypes of dengue virus [49,50], and was a kind gift of Dr. Fernando Arenzana-Seisdedos (Institute Pasteur, Paris). The anti-DIII polyclonal antibody Sango was raised by immunization of a rabbit with purified DENV2 DIII protein [30]. Western blot detection of truncated E proteins used 4G2 or Sango antibodies. A mAb to β-actin was obtained from Sigma (St. Louis, Mo.) and used to confirm equivalent loading of cell lysate samples. Immunofluorescence detection of DENV-infected cells used the antibody to DIII or mouse polyclonal anti-DENV2 hyperimmune ascitic fluid (obtained from Robert B. Tesh, University of Texas Medical Branch), with Alexa Fluor® 488 or rhodamine-conjugated secondary antibodies (Molecular Probes, Life Technologies, Carlsbad, Calif.).

Protein expression, purification, and quantitation: The sequence encoding residues 1-86 of pr was amplified by PCR of an expression plasmid for DENV2 NGC prM-E DI/II [30]. The PCR product was ligated into the pPUR vector (Clontech, Mountain View, Calif.), with the 21-residue TPA signal peptide [65] fused at the N-terminus and a tandem Strep tag at the C terminus (FIG. 1). The plasmid, referred to as pPUR-TPA-pr-STST, was transfected into 293T cells using polyethylenimine (PEI, Polysciences, Warrington, Pa.). For optimal protein production, 3.5×106 cells were plated per 10 cm dish and cultured for 24 h in 10 ml of complete medium. 7.5 μg plasmid in 1 ml DME was mixed with 30 μg PEI, incubated 10 min, then added drop wise to the cell culture medium. After 12 h, the medium was changed to 10 ml DME plus 2% serum. The culture medium was collected after 48 h and again after 72 h. Pr was purified by affinity chromatography on a Strep-Tactin® column from IBA BioTAGnology (Göttingen, Germany) and by gel filtration using a Sephadex® G75 column (Sigma, St. Louis, Mo.) [30]. Final yields were ˜2 mg purified protein/1 liter culture supernatant.

Truncated DENV E proteins (FIG. 1) were obtained by inducible expression in Drosophila S2 cells and purified by affinity chromatography as previously described in detail [29,30]. The H244A mutation was introduced into the DI/II protein by in vitro mutagenesis, and S2 cell expression and purification were performed as above. DENV2 NGC DIII (FIG. 1) was previously referred to as LDIIIH1CS [30], and contains domain III, the linker between domain I and domain III, and the H1 and CS regions of the stem domain. DIII was expressed in E. coli and refolded as previously described [41]. SFV E1 DI/II protein was produced as previously described [29]. All purified proteins were stored in TAN buffer (20 mM Triethanolamine [TEA], pH 8.0; 130 mM NaCl) at −80° C.

SDS-PAGE analysis was performed using 10-12% acrylamide gels with a Bis-Tris buffer system (Invitrogen, now Life Technologies, Carlsbad, Calif.). Western blots were performed with Alexa Fluor® 688-conjugated secondary antibodies (Molecular Probes, Life Technologies, Carlsbad, Calif.), and were quantitated using an Odyssey Infrared Imaging system and Odyssey InCell Western™ software (LI-COR Biosciences, Lincoln, Nebr.) [30]. Standard curves with purified E proteins confirmed the linearity of this analysis (data not shown).

Pull down assay: Pr or BSA was coupled to NHS-activated Sepharose 4™ fast flow (GE Healthcare, Piscataway, N.J.) as described in the manual. In brief, sepharose was washed with 1 mM HCl, and incubated with 660 μg pr or BSA/ml in 0.2 M NaHCO₃, 0.5 M NaCl, pH 8.3 at room temperature for 1.5 hr. The reaction was quenched with 0.1M Tris-HCl pH 8.5 for 30 min and free protein removed by washing with PBS. About 1 mg of protein was coupled to 1 ml beads. For the pull-down assay, 3 μg DI/II protein was pre-incubated where indicated with 24 μg 4G2 (molar ratio 1:2) or control mAb for 10 min at room temperature, and then incubated for 1 h on a rocker at room temperature with 10 μl of pr- or BSA-sepharose in a buffer containing 20 mM MES, 20 mM TEA, 130 mM NaCl, 0.2% Tween® 20 at pH 8.0 or 6.25. The beads were then washed twice with the corresponding buffer and the bound DI/II was analyzed by SDS-PAGE and western blot.

Surface plasmon resonance assays: SPR studies were performed on a BIAcore® 2000 instrument (GE Healthcare, Piscataway, N.J.). Purified recombinant pr was immobilized on a CM5 biosensor chip by primary amine coupling as described in the manual. In brief, pr peptide was diluted to 10 μg/ml in 10 mM sodium acetate pH 4.7 and pre-concentrated on the chip surface. The chip was then activated by a mixture of 1-ethyl-3-(3-dimethylaminopropyl)carbodiimide and N-hydroxysuccinimide, followed by quenching with 1M ethanolamine at pH 8.5. Under these conditions, pr was immobilized to a final density of 600 or 1000 response unit (RU). A control cell was mock-coupled with protein-free solutions. To test interaction, truncated E proteins were diluted to 1.2 mM in a MES/TEA buffer (20 mM MES, 20 mM TEA, 130 mM NaCl) at a pH range of 6.0 to 8.0, and flowed over the chip for 300 s at 0.3 μl/min, followed by buffer alone at the same flow rate. After each round, the chip was regenerated by washing with 50 mM NaOH in 1 M NaCl. The pr chip showed undiminished E binding activity for at least 50 rounds.

Liposome floatation assay: Liposomes were prepared by freeze-thaw and extrusion through 200 nm polycarbonate filters [66], and were stored at 4° C. in TAN buffer under N₂ and used within 2 weeks of preparation. Liposomes were composed of a 1:1:1:3 molar ratio of 1-palmitoyl-2-oleoyl-sn-glycero-3-phosphocholine (POPC), 1-palmitoyl-2-oleoyl-sn-glycero-3-phosphoethanolamine (POPE), sphingomyelin (bovine brain) (Avanti Polar Lipids, Alabaster, Ala.), and cholesterol (Steraloids, Inc., Wilton, N.H.), plus trace amounts of ³H-cholesterol (Amersham, Arlington Heights, Ill.).

Protein-membrane interaction was monitored using a liposome co-floatation assay [29,30]. E′ or DI/II proteins at a final concentration of 50 μg/ml were incubated in TAN buffer (pH 8.0) for 5 min at 28° C. in the presence of 200 μg pr peptide/ml as indicated. Liposomes were then added to a final concentration of 1 mM lipid and the samples were adjusted to pH 5.75 by the addition of 0.3 M MES or maintained at pH 8.0, and the incubation continued at 28° C. for 30-60 min. The samples were then adjusted to 20% sucrose and loaded on top of a 300 μl cushion of 40% sucrose, then overlaid with 1.2 ml 15% sucrose and 200 μl 5% sucrose. All sucrose solutions were at the same pH as the samples, and were wt/wt in TAN buffer at pH 8.0 or in MES buffer (50 mM MES, 100 mM NaCl) at pH 5.5. Gradients were centrifuged for 3 hr at 54,000 rpm at 4° C. in a TLS55 rotor (Beckman Coulter, Inc., Brea, Calif.), and fractioned into the top 700 μl, middle, 400 μl and bottom 1 ml. The ³H-cholesterol marker was quantitated by scintillation counting. 200 μl of each fraction were precipitated with 10% trichloroacetic acid and analyzed by SDS-PAGE and western blotting [29]. Purified human secreted placental alkaline phosphatase with a ST affinity tag (Seap) was used as a control protein [67], and was a kind gift from Yves Durocher, Biotechnology Research Institute, Montreal.

Fusion-infection assay: The fusion-infection assay was performed essentially as described previously [41]. In brief, BfIK cells grown on 96-well plates were washed twice with ice cold binding medium (RPMI without bicarbonate, 0.2% BSA, 10 mM Hepes, and 20 mM NH₄Cl, pH 7.9). Virus stocks were diluted in binding medium and incubated with cells on ice for 3 h with gentle shaking. Cells were washed twice with binding medium to remove unbound virus and pulsed for 1 min at 37° C. in 100 μl RPMI without bicarbonate, containing 0.2% BSA, 10 mM Hepes and 30 mM sodium succinate at pH 6.0 or 7.9, containing the indicated concentration of pr peptide. Infected cells were incubated in MEM plus 2% FCS and 50 mM NH₄Cl for 4 h at 37° C., and then at 37° C. for 2 d in the presence of 20 mM NH₄Cl. The number of infected cells was quantitated by immunofluorescence using mouse polyclonal anti-DENV2 antibody. Infection observed at pH 7.9 represents virus that is endocytosed and fuses during 1 min at this pH.

Generation of DENV1 E H244A mutant: The DENV1-WP infectious clone [reference 68, a kind gift from Dr. Barry Falgout] was digested with KpnI and a 3.3 kb fragment including the E sequence was sub-cloned into the pGEM3Z vector to generate pGDENV1 3.3. pGDENV1 3.3 was used as a template to generate the E H244A mutation, using circular mutagenesis as previously described [69]. A 2.6 kb BstB1/XhoI fragment containing the H244A mutation was sub-cloned into the DENV1-WP infectious clone to obtain DENV1-E H244A. The mutation was confirmed by restriction analysis and sequencing of the complete prM-E region. Two independent infectious clones were used to confirm the phenotype.

The WT and the mutant infectious clones were linearized by Sac II digestion and used as templates for in vitro transcription [70]. RNAs were electroporated into BHK cells and cells were cultured overnight at 37° C. followed by 6 d at 28° C. in MEM containing 2% FBS and 10 mM HEPES, pH 8.0. Progeny virus in the medium was quantitated by infectious center assay on indicator BHK cells, using mouse polyclonal anti-DENV2 antibody. To detect primary infection, aliquots of the electroporated cells were plated on coverslips, cultured 3 d at 37° C., and processed for immunofluorescence microscopy as above.

Expression of prM-E and VLP production. WT and E H244A mutant DENV1 prM-E sequences were PCR-amplified from the pGDENV1 3.3 subclones described above, and cloned into pcDNA4/TO (Invitrogen, Life Technologies, Carlsbad, Calif.). These constructs were transfected into T-REx™-293 cells (Invitrogen, Life Technologies, Carlsbad, Calif.) using Lipofectamine™ 2000 (Invitrogen, Life Technologies, Carlsbad, Calif.) and selected in T-REx HEK medium containing 125 μg/ml Zeocin, all as previous described [61].

To test E protein folding and expression, 1×10⁶ WT and mutant E expressing cells were seeded in 10 cm plates, cultured for 24 h, and then E protein expression was induced by culture for 36 h in 1.5 μg/ml tetracycline in DME medium with 10% FCS at 37° C. Cells were lysed in RIPA buffer (50 mM Tris-HCl pH 7.4, 150 mM NaCl, 1% NP40, 0.5% Na-deoxycholate, 0.1% SDS, 1 mM PMSF, 1× Roche complete protease inhibitor cocktail) on ice for 1 hr. The cell lysates were cleared by centrifugation for 30 min at 10,000×g and protein concentrations were quantitated and normalized. E proteins were immunoprecipitated from cell lysate samples (500 μg total cellular protein) using 20 μg purified mAb 4G2 or mAb 4E11 and 20 μl protein-G sepharose, or 30 μl Sango antibody and 20 μl protein-A sepharose. 4E11 and 4G2 immunoprecipitated samples were blotted with Sango. Sango immunoprecipitated samples were blotted with mouse anti DENV2 serum.

For VLP secretion studies, 2-3×106 cells were seeded in 10 cm plates, cultured for 24 h, and then induced by culture for 36 h in 1.5 μg/ml tetracycline in DME medium with 10% FCS at 37° C. The culture media were centrifuged at 10,000×g for 30 min to remove cell debris. VLPs were then pelleted through a 0.5 ml sucrose cushion by centrifugation at 54,000 rpm for 2 h at 4° C. using a TLS55 rotor. To test the effect of neutralizing the pH of acidic cellular compartments, cells were seeded and induced as above. After 2 h of induction the media were changed to DME medium containing 20 mM HEPES pH 8.0, 2% FCS, and 1.5 μg/ml tetracycline plus 20 mM NH₄Cl as indicated, and the incubation continued for a total of 36 h. E proteins in the cell lysates were immunoprecipitated using mAb 4G2. VLP and lysate samples were then analyzed by SDS-PAGE and western blot using Sango.

### REFERENCES

1. Morens D M, Folkers G K, Fauci A S (2004) The challenge of emerging and re-emerging infectious diseases. Nature 430: 242-249.
2. Mackenzie J S, Gubler D J, Petersen L R (2004) Emerging flaviviruses: the spread and resurgence of Japanese encephalitis, West Nile and dengue viruses. Nat Med 10: S98-109.
3. Weaver S C, Barrett A D (2004) Transmission cycles, host range, evolution and emergence of arboviral disease. Nat Rev Micro 2: 789-801.
4. Lindenbach B D, Thiel H-J, Rice C M (2007) Flaviviruses: the viruses and their replication. In: Knipe D M, Howley P M, editors. Fields' Virology. Fifth ed. Philadelphia: Lippincott, Williams and Wilkins. pp. 1101-1152.
5. Gubler D J (2002) Epidemic dengue/dengue hemorrhagic fever as a public health, social and economic problem in the 21st century. Trends in Microbiol 10: 100-103.
6. Halstead S B (2007) Dengue. Lancet 370: 1644-1652.
7. Kyle J L, Harris E (2008) Global spread and persistence of dengue. Annu Rev Microbiol 62: 71-92.
8. WHO (2009) Dengue and Dengue Haemorrhagic Fever. WHO Fact Sheet N.117.
9. Whitehead S S, Blaney J E, Durbin A P, Murphy B R (2007) Prospects for a dengue virus vaccine. Nat Rev Micro 5: 518-528.
10. Morens D M, Fauci A S (2008) Dengue and hemorrhagic fever: a potential threat to public health in the United States. J Am Med Assn 299: 214-216.
11. Mukhopadhyay S, Kuhn R J, Rossmann MG (2005) A structural perspective of the flavivirus life cycle. Nat Rev Micro 3: 13-22.
12. van der Schaar H M, Rust M J, Waarts B L, van der Ende-Metselaar H, Kuhn R J, et al. (2007) Characterization of the early events in dengue virus cell entry by biochemical assays and single-virus tracking. J Virol 81: 12019-12028.
13. van der Schaar H M, Rust M J, Chen C, van der Ende-Metselaar H, Wilschut J, et al. (2008) Dissecting the cell entry pathway of dengue virus by single-particle tracking in living cells. PLoS Pathog 4: e1000244.
14. Rey F A, Heinz F X, Mandl C, Kunz C, Harrison S C (1995) The envelope glycoprotein from tick-borne encephalitis virus at 2A resolution. Nature 375: 291-298.
15. Modis Y, Ogata S, Clements D, Harrison S C (2003) A ligand-binding pocket in the dengue virus envelope glycoprotein. Proc Natl Acad Sci USA 100: 6986-6991.
16. Modis Y, Ogata S, Clements D, Harrison S C (2005) Variable surface epitopes in the crystal structure of dengue virus type 3 envelope glycoprotein. J Virol 79: 1223-1231.
17. Nybakken G E, Nelson C A, Chen B R, Diamond M S, Fremont D H (2006) Crystal structure of the West Nile virus envelope glycoprotein. J Virol 80: 11467-11474.
18. Kanai R, Kar K, Anthony K, Gould L H, Ledizet M, et al. (2006) Crystal structure of west nile virus envelope glycoprotein reveals viral surface epitopes. J Virol 80: 11000-11008.
19. Zhang Y, Zhang W, Ogata S, Clements D, Strauss J H, et al. (2004) Conformational changes of the flavivirus E glycoprotein. Structure (Camb) 12: 1607-1618.
20. Kuhn R J, Zhang W, Rossman M G, Pletnev S V, Corver J, et al. (2002) Structure of Dengue virus: implications for flavivirus organization, maturation, and fusion. Cell 108: 717-725.
21. Modis Y, Ogata S, Clements D, Harrison S C (2004) Structure of the dengue virus envelope protein after membrane fusion. Nature 427: 313-319.
22. Bressanelli S, Stiasny K, Allison S L, Stura E A, Duquerroy S, et al. (2004) Structure of a flavivirus envelope glycoprotein in its low-pH-induced membrane fusion conformation. EMBO J 23: 728-738.
23. Lescar J, Roussel A, Wien M W, Navaza J, Fuller S D, et al. (2001) The fusion glycoprotein shell of Semliki Forest virus: an icosahedral assembly primed for fusogenic activation at endosomal pH. Cell 105: 137-148.
24. Roussel A, Lescar J, Vaney M-C, Wengler G, Wengler G, et al. (2006) Structure and interactions at the viral surface of the envelope protein E1 of Semliki Forest virus. Structure 14: 75-86.
25. Gibbons D L, Vaney M-C, Roussel A, Vigouroux A, Reilly B, et al. (2004) Conformational change and protein-protein interactions of the fusion protein of Semliki Forest virus. Nature 427: 320-325.
26. Sanchez-San Martin C, Liu C Y, Kielian M (2009) Dealing with low pH: entry and exit of alphaviruses and flaviviruses. Trends in Microbiol 17: 514-521.
27. Harrison S C (2008) Viral membrane fusion. Nat Struct Mol Biol 15: 690-698.
28. Kielian M, Rey F A (2006) Virus membrane fusion proteins: more than one way to make a hairpin. Nat Rev Micro 4: 67-76.
29. Sanchez-San Martin C, Sosa H, Kielian M (2008) A stable prefusion intermediate of the alphavirus fusion protein reveals critical features of class II membrane fusion. Cell Host Microbe 4: 600-608.
30. Liao M, Sanchez-San Martin C, Zheng A, Kielian M (2010) In vitro reconstitution reveals key intermediate states of trimer formation by the dengue virus membrane fusion protein. J Virol 84: 5730-5740.
31. Paroutis P, Touret N, Grinstein S (2004) The pH of the secretory pathway: measurement, determinants, and regulation. Physiology (Bethesda) 19: 207-215.
32. Wengler G (1989) Cell-associated West Nile flavivirus is covered with E+pre-M protein heterodimers which are destroyed and reorganized by proteolytic cleavage during virus release. J Virol 63: 2521-2526.
33. Stadler K, Allison S L, Schalich J, Heinz F X (1997) Proteolytic activation of tick-borne encephalitis virus by furin. J Virol 71: 8475-8481.
34. Li L, Lok S M, Yu I M, Zhang Y, Kuhn R J, et al. (2008) The flavivirus precursor membrane-envelope protein complex: structure and maturation. Science 319: 1830-1834.
35. Yu I M, Holdaway H A, Chipman P R, Kuhn R J, Rossmann M G, et al. (2009) Association of the pr peptides with dengue virus at acidic pH blocks membrane fusion. J Virol 83: 12101-12107.
36. Yu I M, Zhang W, Holdaway H A, Li L, Kostyuchenko V A, et al. (2008) Structure of the immature dengue virus at low pH primes proteolytic maturation. Science 319: 1834-1837.
37. Lorenz I C, Allison S L, Heinz F X, Helenius A (2002) Folding and dimerization of tick-borne encephalitis virus envelope proteins prM and E in the endoplasmic reticulum. J Virol 76: 5480-5491.
38. Lin Y J, Wu S C (2005) Histidine at residue 99 and the transmembrane region of the precursor membrane prM protein are important for the prM-E heterodimeric complex formation of Japanese encephalitis virus. J Virol 79: 8535-8544.
39. Ivy J, Nakano E, Clements D (2000) Subunit immunogenic composition against dengue infection. U.S. Pat. No. 6,165,477.
40. Junjhon J, Lausumpao M, Supasa S, Noisakran S, Songjaeng A, et al. (2008) Differential modulation of prM cleavage, extracellular particle distribution, and virus infectivity by conserved residues at nonfurin consensus positions of the dengue virus pr-M junction. J Virol 82: 10776-10791.
41. Liao M, Kielian M (2005) Domain III from class II fusion proteins functions as a dominant-negative inhibitor of virus-membrane fusion. J Cell Biol 171: 111-120.
42. Dejnirattisai W, Jumnainsong A, Onsirisakul N, Fitton P, Vasanawathana S, et al. (2010) Cross-reacting antibodies enhance dengue virus infection in humans. Science 328: 745-748.
43. Kroschewski H, Sagripanti J L, Davidson A D (2009) Identification of amino acids in the dengue virus type 2 envelope glycoprotein critical to virus infectivity. J Gen Virol 90: 2457-2461.
44. Allison S L, Stadler K, Mandl C W, Kunz C, Heinz F X (1995) Synthesis and secretion of recombinant tick-borne encephalitis virus protein E in soluble and particulate form. J Virol 69: 5816-5820.
45. Lorenz I C, Kartenbeck J, Mezzacasa A, Allison S L, Heinz F X, et al. (2003) Intracellular assembly and secretion of recombinant subviral particles from tick-borne encephalitis virus. J Virol 77: 4370-4382.
46. Hsieh S C, Liu I J, King C C, Chang G J, Wang W K (2008) A strong endoplasmic reticulum retention signal in the stem-anchor region of envelope glycoprotein of dengue virus type 2 affects the production of virus-like particles. Virol 374: 338-350.
47. Corver J, Ortiz A, Allison S L, Schalich J, Heinz F X, et al. (2000) Membrane fusion activity of tick-borne encephalitis virus and recombinant subviral particles in a liposomal model system. Virol 269: 37-46.
48. Wang P G, Kudelko M, Lo J, Siu L Y, Kwok K T, et al. (2009) Efficient assembly and secretion of recombinant subviral particles of the four dengue serotypes using native prM and E proteins. PLoS One 4: e8325.
49. Lisova O, Hardy F, Petit V, Bedouelle H (2007) Mapping to completeness and transplantation of a group-specific, discontinuous, neutralizing epitope in the envelope protein of dengue virus. J Gen Virol 88: 2387-2397.
50. Thullier P, Lafaye P, Megret F, Deubel V, Jouan A, et al. (1999) A recombinant Fab neutralizes dengue virus in vitro. J Biotechnol 69: 183-190.
51. Crill W D, Chang G J (2004) Localization and characterization of flavivirus envelope glycoprotein cross-reactive epitopes. J Virol 78: 13975-13986.
52. Konishi E, Mason P W (1993) Proper maturation of the Japanese encephalitis virus envelope glycoprotein requires cosynthesis with the premembrane protein. J Virol 67: 1672-1675.
53. Demaurex N, Furuya W, D'Souza S, Bonifacino J S, Grinstein S (1998) Mechanism of acidification of the trans-Golgi network (TGN). In situ measurements of pH using retrieval of TGN38 and furin from the cell surface. J Biol Chem 273: 2044-2051.
54. Zhang X, Fugere M, Day R, Kielian M (2003) Furin processing and proteolytic activation of Semliki Forest virus. J Virol 77: 2981-2989.
55. Stiasny K, Allison S L, Schalich J, Heinz F X (2002) Membrane interactions of the tick-borne encephalitis virus fusion protein E at low pH. J Virol 76: 3784-3790.
56. Fritz R, Stiasny K, Heinz F X (2008) Identification of specific histidines as pH sensors in flavivirus membrane fusion. J Cell Biol 183: 353-361.
57. Nelson S, Poddar S, Lin T Y, Pierson T C (2009) Protonation of individual histidine residues is not required for the pH-dependent entry of west nile virus: evaluation of the “histidine switch” hypothesis. J Virol 83: 12631-12635.
58. Schmidt A G, Yang P L, Harrison S C (2010) Peptide inhibitors of dengue-virus entry target a late-stage fusion intermediate. PLoS Pathog 6: e1000851.
59. Kaufmann B, Chipman P R, Holdaway H A, Johnson S, Fremont D H, et al. (2009) Capturing a flavivirus pre-fusion intermediate. PLoS Pathog 5: e1000672.
60. Vashishtha M, Phalen T, Marquardt M T, Ryu J S, Ng A C, et al. (1998) A single point mutation controls the cholesterol dependence of Semliki Forest virus entry and exit. J Cell Biol 140: 91-99.
61. Taylor G M, Hanson P I, Kielian M (2007) Ubiquitin depletion and dominant-negative VPS4 inhibit rhabdovirus budding without affecting alphavirus budding. J Virol 81: 13631-13639.
62. Umashankar M, Sanchez San Martin C, Liao M, Reilly B, Guo A, et al. (2008) Differential cholesterol binding by class II fusion proteins determines membrane fusion properties. J Virol 82: 9245-9253.
63. Thomas J M, Klimstra W B, Ryman K D, Heidner H W (2003) Sindbis virus vectors designed to express a foreign protein as a cleavable component of the viral structural polyprotein. J Virol 77: 5598-5606.
64. Stiasny K, Kiermayr S, Holzmann H, Heinz F X (2006) Cryptic properties of a cluster of dominant flavivirus cross-reactive antigenic sites. J Virol 80: 9557-9568.
65. Wang P, Chen J, Zheng A, Nie Y, Shi X, et al. (2004) Expression cloning of functional receptor used by SARS coronavirus. Biochem Biophys Res Comm 315: 439-444.
66. Chatterjee P K, Vashishtha M, Kielian M (2000) Biochemical consequences of a mutation that controls the cholesterol dependence of Semliki Forest virus fusion. J Virol 74: 1623-1631.
67. Cass B, Pham P L, Kamen A, Durocher Y (2005) Purification of recombinant proteins from mammalian cell culture using a generic double-affinity chromatography scheme. Protein Expr Purif 40: 77-85.
68. Puri B, Polo S, Hayes C G, Falgout B (2000) Construction of a full length infectious clone for dengue-1 virus Western Pacific,74 strain. Virus Genes 20: 57-63.
69. Chanel-Vos C, Kielian M (2004) A conserved histidine in the ij loop of the Semliki Forest virus El protein plays an important role in membrane fusion. J. Virol 78: 13543-13552.
70. Liljeström P, Lusa S, Huylebroeck D, Garoff H (1991) In vitro mutagenesis of a full-length cDNA clone of Semliki Forest virus: the small 6,000-molecular-weight membrane protein modulates virus release. J Virol 65: 4107-4113.
71. K Irie et al., Sequence analysis of cloned dengue virus type 2 genome (New Guinea-C strain), Gene, Volume 75, Issue 2, 20 February 1989, Pages 197-211

1. A method for treating or preventing infection by a flavivirus of a cell in a subject comprising administering to the subject an amount at an agent effective to (i) inhibit interaction of a pr peptide of the flavivirus and a membrane fusion protein r (E protein) of the flavivirus, or (ii) inhibit interaction between the E protein of the flavivirus and the membrane of the cell.

2. A method for determining if an agent can prevent or reduce exocyrosis from a flavivirus-infected cell of a flavivirus virus synthesized by the cell comprising:
a) contacting membrane fusion protein E (“E protein”), or portion thereof, of the flavivirus with (i) a flavivirus pr peptide, and (ii) the agent under conditions permitting the pr peptide to bind to the E protein or portion thereof; and
b) quantifying the binding between the pr peptide and the F protein or portion thereof,
wherein a decrease in the binding between the pr peptide and the F protein in the presence of the agent relative to binding between the pr peptide and the E protein in the absence of the agent indicates that the agent can prevent or reduce exocyrosis of the flavivirus virus synthesized by the cell from the flavivirus-infected cell, while no change in or an increase in the binding between the pr peptide and the E protein in the presence of the agent relative to binding between the pr peptide and the E protein in the absence of the agent indicates that the agent is not useful to prevent or reduce exocyrosis of the flavivirus virus from the flavivirus-infected cell.

3. A method for determining if an agent can reduce or prevent fusion of a flavivirus with a cell membrane comprising:
a) contacting a membrane fusion protein E (“E protein”), or portion thereof, of the flavivirus with a flavivirus pr peptide and the agent under conditions permitting the pr peptide to bind to the E protein or portion thereof; and
b) quantifying the binding between the pr peptide and the E protein or portion thereof,
wherein an increase in the binding between the pr peptide and the E protein in the presence of the agent relative to binding of the pr peptide and the E protein in the absence of the agent indicates that the agent can reduce or prevent fusion of the flavivirus with the cell membrane, while no change in or a decrease in the binding between the pr peptide and the E protein in the presence of the agent relative to binding between the pr peptide and the E protein in the absence of the agent indicates that the agent is not useful to reduce or prevent fusion of flavivirus with a cell membrane.

4. The method of claim 2, wherein the agent is membrane-permeable.

5. The method of claim 3, wherein the agent is membrane-impermeable.

6. The method of claim 1, wherein the agent is an organic molecule having a molecular weight of 800 daltons or less.

7. The method of claim 2, wherein the portion of the E protein is contacted with the flavivirus pr peptide and the agent, and wherein the portion of the E protein comprises E protein domain I and E protein domain II.

8. The method of claim 2, wherein the portion of the E protein is derived from a Dengue virus.

9. The method of claim 8, wherein the Dengue virus is a Dengue Virus 2.

10. (canceled)

11. The method of claim 2, wherein the flavivirus is a Dengue virus.

12. The method of claim 1, wherein the agent effective to inhibit interaction between the E protein of the flavivirus and the membrane of the cell is an isolated pr peptide or an isolated fragment of a pr peptide.

13. The method of claim 12, wherein the isolated pr peptide or the isolated fragment of a pr peptide is derived from a Dengue virus.

14. The method of claim 13, wherein the isolated pr peptide or the isolated fragment of a pr peptide is derived from a Dengue virus New Guinea C strain.

15. The method of claim 1, wherein the agent comprises residues 1-86 of the prM-E protein of Dengue Virus 2 New Guinea C strain.

16. (canceled)

17. The method of claim 2, wherein the portion of the E protein is contacted with the flavivirus pr peptide, and the portion of the E-protein is a soluble truncated E-protein or a Dengue virus 2 ij loop having the sequence set forth in SEQ ID NO:4.

18. The method of claim 2, wherein the conditions permitting the pr peptide to bind to the E protein or portion thereof comprise an acidic pH.

19. The method of claim 3, further comprising, when an agent is found to increase the binding of the pr peptide to the E protein, determining if the agent increases the binding of the pr peptide to the E protein at a neutral pH.

20. A peptide comprising consecutive amino acid residues having the sequence TFKNPHAKKQDVVV (SEQ ID NO:4).

21. The peptide of claim 20, wherein the peptide is GCTFKNPHAKKQDVVVC (SEQ ID NO:5).

22. A pharmaceutical composition comprising the peptide of claim 20 or 21 and a pharmaceutically acceptable carrier.

23-30. (canceled)
